(19) United States
(12) Patent Application Publication
(10) Pub. No.: US 2020/0054657 A1
(43) Pub. Date: Feb. 20, 2020

(54) Compositions of Matter and Methoods to Treat Cancer

(71) Applicants: Thomas E Adrian, Oak Park, IL (US); Peter D Collin, Deer Isle, ME (US); Robert A Newman, Surry, ME (US)

(72) Inventors: Thomas E Adrian, Oak Park, IL (US); Peter D Collin, Deer Isle, ME (US); Robert A Newman, Surry, ME (US)

(21) Appl. No.: 15/999,295

(22) Filed: Aug. 20, 2018

(51) Int. Cl. A61K 31/704; A61K 31/341; A61P 35/00; A61K 9/48

(52) U.S. Cl. CPC A61K 31/704; A61K 9/4891; A61P 35/00; A61K 31/341

(57) ABSTRACT

The named inventors claim discovery of synergistic anti-cancer effects of combinations of one or more sea cucumber glycosides such as Frondoside A or other sea cucumber glycosides and Andrographolide or an Andrographolide analog, wherein the biological effects are may be more beneficial than the single components making up the said invention. The inventors claim new methods and compositions of matter to treat cancer in mammals, and additionally a method to effectively treat cancer stem cells in mammals, with a composition comprised of one or more sea cucumber glycosides and Andrographolide, or an andrographolide analog.

The inventors have discovered that a triterpenoid glycoside from the Atlantic sea cucumber (Cucumaria frondosa) called Frondoside A, that potently inhibits proliferation of multiple types of cancer (including solid cancers such as breast, pancreas, colon, lung, prostate, and leukemias) can be surprisingly combined with Andrographolide to effectively make the combination be a synergistic new treatment method and composition of matter with improved anti-cancer effects in vivo with total or almost total inhibition of cancer cell proliferation. Frondoside A is known to block the cell cycle and cause programmed cell death (apoptosis) in several cancers. In addition, Frondoside A inhibits cell invasion, angiogenesis (formation of new blood vessels to supply the tumor) and development of metastases in distant tissues. Combination of Frondoside A with Andrographolide shows an unexpected significant synergistic enhanced anticancer effect compared with either of the single drugs alone. Since this is seen in both leukemias and in pancreatic adenocarcinomas (solid tumors) this appears to be a general phenomena for many cancers. The finding is remarkable, since on its own, Andrographolide has only mediocre inhibitory effects on cancer cell proliferation.

Using the therapeutic combinations described herein, the inventors are for the first time able to completely eradicate all the cancer cells in some cell lines. This indicates that all the cancer stem cells as well as the large population that makes up the tumor are being killed. This is important since current cancer therapies do not eradicate cancer stem cells. Furthermore, since cancer stem cells are the only cells that can recapitulate a tumor, their eradication is essential for combatting the disease.

A topical preparation was made comprised of a mixture of Frondoside A, Frondoside B and Frondoside C in an approximate ratio of 60:20:20, respectively, from a column chromatographic separation of the total glycosides of Cucumaria frondosa ethanol/water extraction wash (70:30) and mixed with an Andrographolide-containing extract derived from a 10% Andrographolide powder by an ethanol extraction and concentrated (Andrographolide crude extract approximately 80% andrographolide). The combination of sea cucumber glycosides and the crude Andrographolide extract were mixed at 80:20% respectively and were further mixed into a cosmetic carrier comprised of lanolin and beeswax with 0.2% DMSO, with the total Frondoside glycosidic/Andrographolide fraction being approximately 3% of the total composition, and applied to distinct actinic keratosis lesions on a patient's forehead twice per day for 6 days. After 6 days, actinic keratosis lesions were visibly decreased by approximately 70%. Continued application of the cosmetic carrier containing the Frondoside and Andrographolide crude extracts on a daily basis for a total of 14 days resulted in complete disappearance of the actinic keratosis lesions.

The inventors claim non-obvious discoveries of the combination of Frondoside A or Frondoside B or other structurally related sea cucumber glycosides with Andrographolide or crude andrographolide compounds as having a synergy of inhibitory effects on cancer cell proliferation, and by extension, which can be further developed more specifically by known “drug development methodologies” by those skilled in the arts. Additionally, the inventors of the present invention claim compositions of matter comprised of Frondoside A and/or other sea cucumber glycosides with Andrographolide specifically that can eradicate cancer stem cells and be valuable in the treatment of cancer in mammals as intraperitoneal injections, orally administered as enteric buffered or unbuffered capsules, topically, buccal, intravenously, intraperitoneally, and by suppository medicines or other delivery methods as are commonly known to those skilled in the arts.

The present invention is in the technical field of cancer treatment in mammals. More particularly, the present invention is in the technical field of treating human cancers by compositions of matter incorporating synergistic inhibitors of biological pathways in mammals wherein the combinations are more effective than the individual components for the treatment and prevention of cancer.

More particularly, the present invention relates to and involves an unexpected synergy between Frondoside A and/or sea cucumber glycosides of similar structure and Andrographolide or other Andrographis-derived compounds or synthetically produced, for the treatment of pre-cancers and cancers in mammals. More particularly, the present invention relates to individual sea cucumber glycosides or mixtures of different sea cucumber glycosides that when mixed with Andrographolide or Andrographolide-compounds, inhibit cancer cell growth and tumor proliferation in mammals. More particularly, the present invention is concerned with the improvement of clinical effectiveness by the use of one or more sea cucumber glycosides such as Frondoside A when used in conjunction with Andrographolide. The combination consists of between 0.1% to 99.9% glycoside and between 0.1% to 99.9% Andrographolide or andrographolide compound, depending on formulation/effectiveness based on parameters developed by those skilled in the arts.

Sea Cucumber Anti-Cancer Glycosides

Sea cucumber glycosides are described and reviewed in (Mar. Drugs 2015, 13(3), 1202-1223.—Review—Anticancer Activity of Sea Cucumber Triterpene Glycosides), and are included herein by reference. Additionally, US patents by Collin, et al, are included herein by reference, and internationally, foreign patents by Aminin, et al are included herein by reference showing sea cucumber glycosides as anti-cancer agent art, and are included herein by reference. The existing research about and with sea cucumber glycosides has not thus far shown total (100%) inhibition of cancer proliferation in an animal nor total inhibition of all cancer cells in vitro, including cancer stem cells. Therefore, it is advantageous to find a new method for treating cancer and to produce compositions of matter wherein the existing anti-cancer activity of sea cucumber glycosides are potentiated and are able to become effective to a human or other animal by decreasing the subjects' cancer load and especially targeting cancer stem cells, which is an object of the present invention. The inventors propose new compositions of matter and methods for cancer treatment and prevention comprising and composed of, combinations of sea cucumber glycosides and Andrographolide compounds suitable for dosing to a subject in need and administered in a manner appropriate to the needs of the subject, as are known by those skilled in the arts. It was surprisingly found by the inventors that the addition of Andrographolide was synergistic in its anti-cancer effects, rather than additive in its anti-cancer effects when combined with a sca cucumber glycoside, and more specifically, Frondoside A.

The inclusion of one or more sea cucumber glycosides, other than Frondoside A or B or C (See FIG. 1.) with an Andrographolide (See FIG. 2.) compound is contemplated to exert increased synergistic inhibition of cancer stem cells better than either drug alone, as discovered with Frondoside A when complexed with Andrographolide. The drug or drugs of the present invention can be given topically, transdermally, buccally, per os, intraperitoneally, rectally or intravenously, or in enteric coated capsules in a dosing regimen based on method developments known to those skilled in the arts.

There is a well-known need within the cancer treatment and pharmaceutical industry to improve and discover additional anti-cancer compounds and to discover synergies with existing or future anti-cancer compounds that effectively dampen, control, inhibit and/or prevent the spread of cancers of various kinds.

Background of Andrographis and Andrographolide

Andrographis (Andrographis paniculata (Burm.f.) Wall. Nees. Family: Acanthaceae) is also known as Kalmegh (Hindi), Kalmegha (Snaskrit), Chuanxinlian (Chinese), Kalupnath , Kirayat (Hindi), Mahatita (King of Bitters), Alui , Bhunimba , Bhui-neem , Yavatikta (Sanskrit), Sam biloto (Malay). This plant or extracts of it has been used for liver complaints and fever, and as an anti-inflammatory and immunostimulant. In clinical trials, Andrographis extract has been studied for use as an immunostimulant in upper respiratory tract infections and HIV infection. The potential of Andrographolide as an anticancer agent is being investigated.

Andrographis has been used for centuries in India, China, Thailand, and other Asian countries and is present in 26 different polyherbal formulations in the Ayurvedic traditional health system. Kalmegh is listed in the 1992 Pharmacopoeia of the Peoples Republic of China as a cold property herb used to rid the body of fevers and dispel toxins. An immunostimulant preparation known as Kan Jang , which contains Kalmegh and eleutherococcus, has been used in Scandinavian countries for 20 years.

The diterpene lactone andrographolide was first isolated as a major constituent and later characterized as a lactonc. Its full structure was determined in the 1960s, and x-ray crystallography later confirmed the structure. A number of related minor diterpenes and their glycosides have since been identified. Methods of analysis, including high-pressure liquid chromatography and nuclear magnetic resonance, have been published. A method for rapid isolation of Andrographolide is also available. When callus cultures of the plant were investigated, Andrographolide and the other diterpenes were not produced. Instead, the sesquiterpenes paniculides A-C were found. Other constituents of the plant include various flavones.

Animal and in vitro experiments using human cancer cell lines to investigate the potential anticancer effects of A. paniculata have found Andrographolide responsible for the observed effects rather than other diterpenes. Various mechanisms of action have been proposed, including enhancement of chemokine activity, inhibition of tumor-specific angiogenesis affecting cell cycle progression, and induction of apoptosis. Cancer cell lines investigated include prostate, breast, cervical, colon, hepatoma, melanoma, and lymphocytic leukemia.

### SUMMARY OF THE INVENTION

The present invention improves and advances anti-cancer methods and compositions known in the medical literature, by combining one or more sea cucumber glycosides that may include Frondoside A and/or Frondoside B, and an Andrographolide compound, preferably Andrographolide for oral, IP, IV, topical, transdermally, buccal or rectal administration of a medicament comprised of said sea cucumber glycoside(s) and said Andrographolide compounds, The inventors have unexpectedly found that there is a potent synergy of anti-cancer effects, including diminishment of cancer stem cells as are commonly known, by administration to a subject in need of anti-cancer medicines consisting of a combination of one or more sea cucumber glycosides and an andrographolide compound. Frondoside A and some other sea cucumber specific glycosides are known inhibitors of various cancers. The inventors have surprisingly found that a beneficial synergy is obtained by the addition of Andrographolide with Frondoside A and because of the similarities shared by all sea cucumber glycosides, claim that other sea cucumber glycosides, besides Frondoside A will have similar synergies with Andrographolide. Specific combinations that promote this beneficial synergy and morc specifically, a synergy between Frondoside A are discovered by the inventors to be inhibitory of certain types of cancer stem cells in various cancers, with near complete inhibition of total proliferating cancer cells of various types as are commonly known to persons skilled in the cancer treatment arts. Additionally, the inventors have found that the combination of sea cucumber glycosides, including Frondoside A and or Frondoside B, provide a synergy of anti-cancer effects more than the individual components alone.

The observations that this works in both acute leukemias and in pancreatic cancer suggests a generalized phenomenon and that these treatment combinations should be effective in many cancers.

### BRIEF DESCRIPTIONS OF THE DRAWINGS

FIG. 1. Frondosides A, B and C structures

FIG. 2. Andrographolide structure

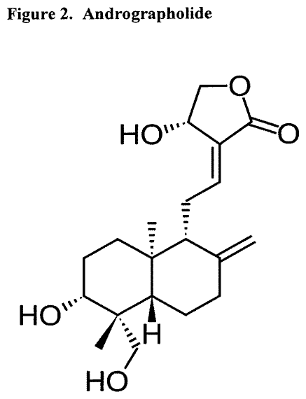

FIG. 3. Andrographolide (50 μM) markedly enhances the effect of low doses of Frondoside A on viability of the two human, acute leukemia cell lines CCRF-CEM and THP-lindicating that Andrographolide enhances the effects of Frondoside A in human acute leukemia blast cells and in the THP-1 cells, any existing cancer stem cells present. Andrographolide (50 μM) alone reduced cell viability to 54.3±2.8% in CCRF-CEM cells and to 68.5±1.4%. *** =P<0.001.

FIG. 4. Andrographolide markedly enhances the effect of low doses of Frondoside A on viability of the two human, pancreatic cancer cell lines, AsPC-1 and S2013. This indicates that Andrographolide also enhances the effects of Frondoside A in human pancreatic cancer cells, and in this AsPC-1 assay, kills cancer stem cells. This suggests a general phenomenon applicable to all or many cancers. * * * =P<0.001.

FIG. 5. Frondoside A was recently shown to be an inhibitor of PAK-1 kinase [Nguyen BCQ et al.] Drug Disc Therapeut 2017; 11(2): 110-114. Here we compare the effects of Frondoside A and two known PAK-1 inhibitors FRAX597 and FRAX1036 (and the PAK-3 inhibitor PF3758309) on viability of AsPC-1 human pancreatic cancer cells. All three PAK-1 inhibitors show similar inhibitory effects on cell viability. The fact that FRAX597 and FRAX1036 are more potent inhibitors of PAK-1 activity in vitro, but arc less potent anti-cancer compounds is explained by their relative insolubility and cell membrane penetrations.

FIG. 6. Two PAK-1 inhibitors, FRAX597 and FRAX1036 cause dose responsive reduction in viability of AsPC-1 human pancreatic cancer cells. However, there is no enhancing effect of Andrographolide on the FRAX597 and FRAX1036 responses. Indeed, there is a tendency of the reduced effectiveness of FRAX597 in the presence of Andrographolide.

FIG. 7. Two PAK-1 inhibitors, FRAX597 and FRAX1036 cause modest dose responsive reduction in viability of HT29 human colonic cancer cells. However, there is no enhancing effect of Andrographolide on the FRAX597 and FRAX1036 responses. Indeed, there is a tendency of the reduced effectiveness of FRAX597 in the presence of Andrographolide.

### INTRODUCTION

Development of novel therapeutics for cancer is essential if we are to improve the long-term outcome for patients, particularly those with metastatic disease. While late diagnosis undoubtedly contributes to the lack of response of these cancers to therapy, it has become apparent that the presence of cancer stem cells (CSC), which are not easily eradicated by standard chemotherapeutics, are ultimately responsible for the failure of existing strategies [1-2]. Recent understanding of the heterogeneous makeup of the cancer cells in a tumor revealed the presence of CSCs [3-5]. CSCs can be characterized their self-renewing ability via asymmetric division, ability to differentiate into diverse phenotypes, ability to initiate tumors from minute numbers, and chemoresistance [4-5]. As expected, the discovery of CSCs in cancer development has reshaped our understanding of cancer biology and is revolutionizing the current efforts of developing new therapeutics. Recent studies have demonstrated the unique properties of CSCs in many tumor types, including breast, colon, melanoma, brain, bone, ovary, prostate, pancreatic and liver cancers [1-3]. In leukemia, the mutated stem cells result in an accumulation of immature progenitors, known as blast cells. By FACS analysis, Li et al isolated CD44+/CD24+/EpCAM+(CD326) pancreatic CSCs which counted for 0.2-0.8% of total cancer cells, showed stem-cell like properties and had the capability of form tumors in animal models when as few as 100 cells [1]. Hermann et al characterized another subpopulation of pancreatic CSCs expressing the surface marker CD133, and described it to be exclusively tumorigenic and highly resistant to standard chemotherapy [2]. Functional assays such as screening cells with a high activity of aldehyde dehydrogenase could also be used to identify CSCs [3]. Being a viable cause of metastasis along with their high resistance to conventional chemotherapy has led to the growing belief that CSCs can be the ultimate foe in the combat against cancer. It is common wisdom that therapies that would specifically eradicate those CSCs are being actively investigated [6-10]. CSCs are known to contribute to tumor initiation, self-renewal, chemoresistance, and metastasis [5]. Indeed, they are the only cells from a tumor able to recapitulate the disease, indicating that their eradication is essential [5].

Multiple developmental pathways involved in growth and differentiation are important for function of CSCs and thus provide possible targets for their eradication. These include the Hedgehog, notch, CXCR4, nuclear factor kappa B (NF-κB) and Wnt/β-catenin (Wnt) pathways. [13].

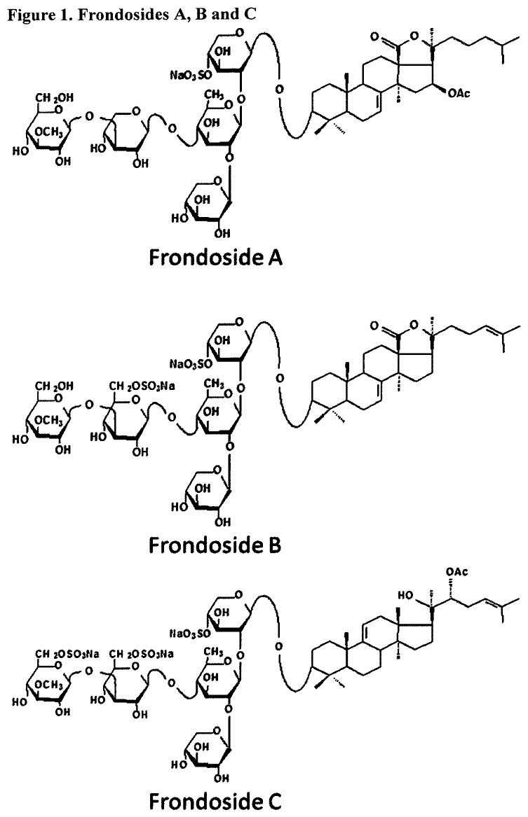

Frondoside A (See FIG. 1.) is a triterpenoid glycoside isolated from the edible sea cucumber Cucumaria frondosa. Frondoside A has been shown to be an effective anti-cancer agent in several solid malignancies, including pancreatic, colonic, breast, prostate and lung cancers, with induction of apoptosis, as evidenced by morphological changes, annexin V binding, upregulation of the cyclin-dependent kinase inhibitor p21^{waf1}, upregulation of proapoptotic proteins such as Bax, downregulation of antiapoptotic proteins such as Mcl-1 and Bcl2, activation of caspase 3, 6 and 9 activities and TUNEL assay [14-18]. Frondoside A inhibited cell proliferation and induced apoptosis in two myeloid leukemia cell lines [19]. Frondoside A also inhibits invasion, metastases and angiogenesis in several models [14-18], potentiates the effects of other therapeutic agents [16, 20], reduces multidrug resistance [21], and shows anti-inflammatory and immunomodulatory effects [22]. Frondoside A is well-tolerated, does not have toxic effects at therapeutic doses, as evidenced by lack of change in CBC, liver function tests, body weight, etc. [15, 16], and has an LD₅₀ of 9.9 mg/kg in mice, which is more than 1000 times higher than the dose used therapeutically in this species. Its anticancer effect is not tissue specific and this makes it a potential treatment for many types of malignant diseases. Frondoside A was recently shown to be an inhibitor of PAK-1 kinase [23].

### Supporting Observations

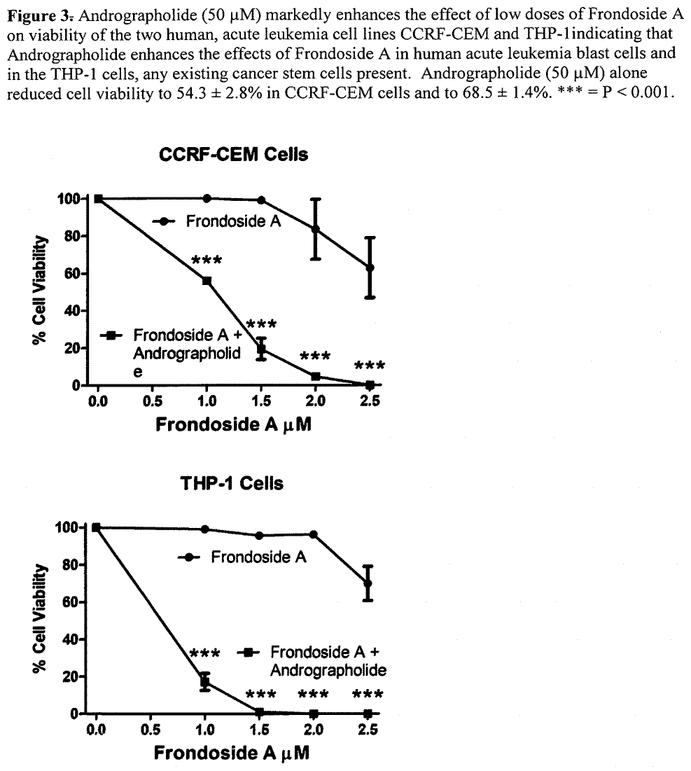

It has been surprisingly found by the inventors that Andrographolide (50 μM) markedly enhances the effect of low concentrations of Frondoside A on viability of the two, human, acute leukemia cell lines CCRF-CEM and THP-1. This indicates that Andrographolide enhances the effects of Frondoside A in human acute leukemia blast cells. See FIG. 3. Because of the total abrogation of THP-1 cells, it is reasonable to claim that this combination of sea cucumber glycoside and an Andrographolide compound is inhibitory of cancer stem cells.

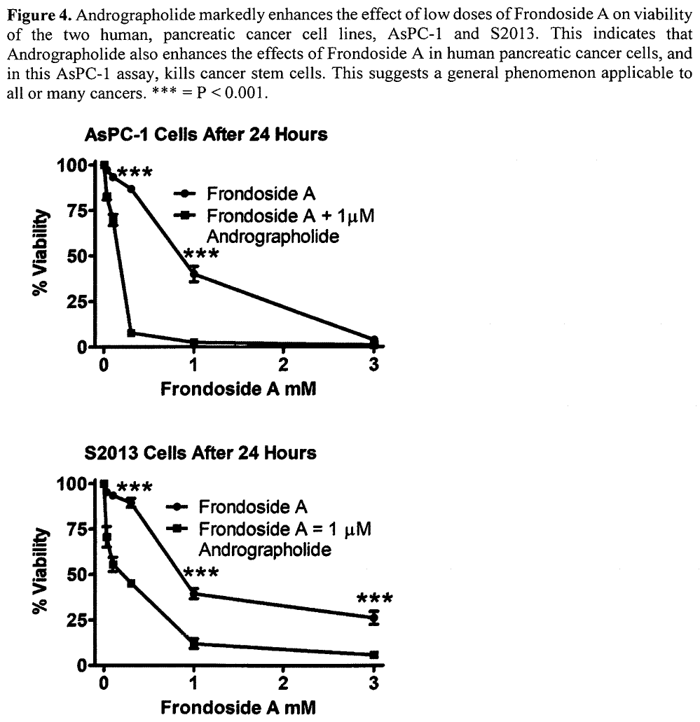

The inventors have additionally and unexpectedly found that Andrographolide markedly enhances the effect of low concentrations of Frondoside A on viability of the two human, pancreatic cancer cell lines, AsPC-1 and S2013. This indicates that Andrographolide also enhances the effects of Frondoside A in human pancreatic cancer cells. This suggests a general phenomenon applicable to other cancers. See FIG. 4.

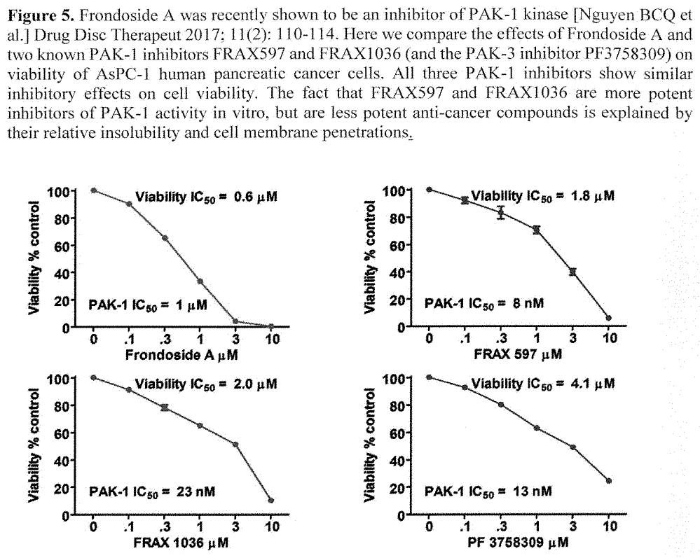

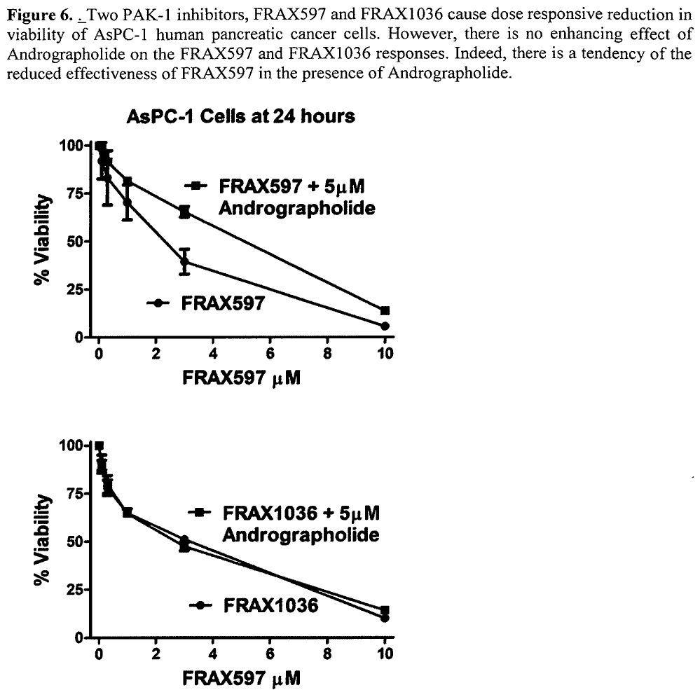

Frondoside A was recently shown to be an inhibitor of PAK-1 kinase [Nguyen BCQ et al. Drug Disc Therapeut 2017; 11(2):10-114]. We have compared the effects of Frondoside A and two known PAK-1 inhibitors FRAX597 and FRAX1036 (and the PAK-3 inhibitor PF3758309) on viability of AsPC-1 human pancreatic cancer cells. All three PAK-1 inhibitors show similar inhibitory effects on cell viability. The fact that FRAX597 and FRAX1036 are more potent inhibitors of PAK-1 activity in vitro, but are less potent anti-cancer compounds is explained by their relative insolubility and limited cell membrane penetrations. See FIGS. 5 and 6.

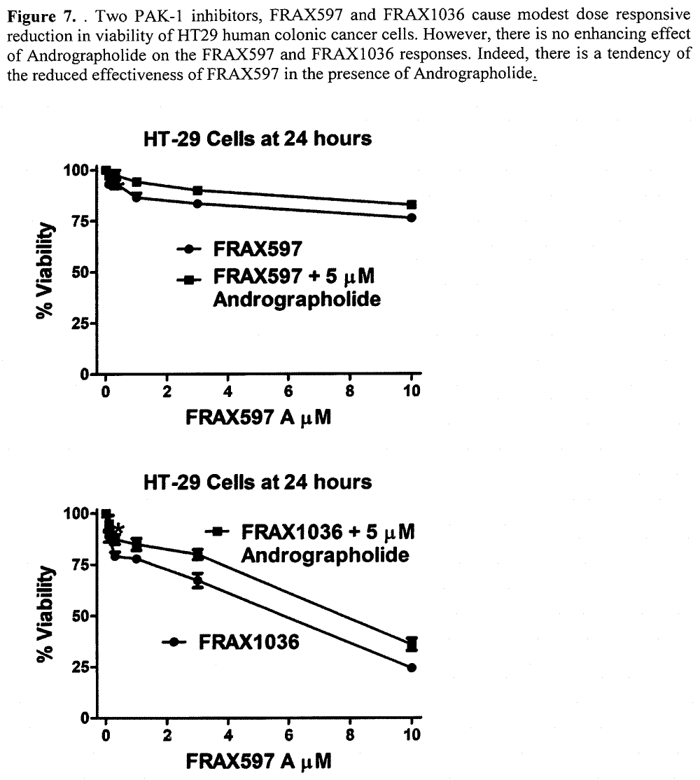

Two PAK-1 inhibitors, FRAX597 and FRAX1036 cause modest dose responsive reduction in viability of HT29 human colonic cancer cells. However, there is no enhancing effect of Andrographolide on the FRAX597 and FRAX1036 responses. Indeed, there is a tendency toward reduced effectiveness of FRAX597 in the presence of Andrographolide. See FIG. 7.

Additionally, it is contemplated that other sea cucumber glycosides mixed with Andrographolide or neoandrographolide, 12-deoxy-11,12-didehydroandrographolide, deooxyandrographolide, andrographiside, andrograpanin, or dehydroandrographolide in effective concentrations known to those skilled in the arts, will act in a similar fashion synergistically against cancer cell proliferation and/or cancer stem cell activation and proliferation, and are included herein by reference.

In an additional embodiment of the invention, a drug is provided to a patient by a person skilled in the arts, that is a combination of Frondoside A and Andrographolide or an Andrographolide compound in proportions between 0.1%: 99.9% Frondoside A to Andrographolide or 90.9% Frondoside A to 0.1% Andrographolide, wherein the effect of said invention is the more complete inhibition of cancer cells in an animal and/or cancer stem cells in a mammal than by either of the individual components alone.

### Other Sea Cucumber Glycosides Useful in the Present Invention

In an additional embodiment, the invention would be a composition of matter with an andrographolide compound, a sea cucumber glycoside chosen from the list that includes:

1. Cucumarioside A2-2, A1, A3, A4, A5, A6, A12, and A15

2. Cucumarioside H3, H4

3. Stichoposide C

4. Okhotoside B3

5. Okhotoside B2, B1

6. Intercedenside A

7. Colochiroside A

8. 24-dehydro echinoside

9. Echinoside A

10. Ds-echinoside A

11. Holothurin A

12. Patagonicoside A

13. Philinopside E

14. Frondoside C

15. Pilinopside A and B

16. Bivittoside A

17. Hillaside B

18. Bivittoside A,

19. Scabraside D

20. fuscocineroside C

21. 24-dehydroechinoside A

22. Cucumarioside H3,

23. 3-O-methyl-D-xylose, Hillaside A,

24. 17-dehydroxyholothurinoside Am,

25. Griseaside A

26. Griseaside D1, Dx and E

27. Philinopgenin A

28. Philinopgenin C

29. Peulactasides 1,11, 111

30. Violaceusides A and B

31. Stichorrenoside C and B

32. Philinopgenin B

33. Synallactosides A1, A2, B1, B2 and C

The sea cucumber glycoside chosen from the list above is combined with an Andrographolide compound in proportions between 0.1%: 99.9% of the total and the sea cucumber glycoside from between 0.1% to 99.9% of the total wherein the effect of said combination of glycoside to Andrographolide exerts more complete inhibition of cancer cells in a mammal and/or cancer stem cells in a mammal, than by either of the individual components alone.

Said composition of matter comprising said glycoside chosen from the list of sea cucumber glycosides combined with an Andrographolide compound has minimal side effects, and is administered to a mammal by oral, intraperitoneal, intravenous, topical or buccal or rectal methods. Compositions of the present invention comprising an anti-cancer sea cucumber glycoside complexed with Andrographolide are administered intravascularly, intraperitoneally, orally, intraperitoneally, subcutaneously, intramuscularly, or by intramammary infusion using forms known in the pharmaceutical art. For intravascular, intraperitoneal, subcutaneous, intramuscular administration methods, active drug components are combined with a suitable carrier such as water, saline, aqueous dextrose, and the like. Veterinary formulations comprising sea cucumber glycoside, preferably Frondoside A or Frondoside B complexed with Andrographolide are prepared by mixing said synergistic complex with an excipient, diluting by an excipient or enclosing within a carrier that can be in the form of a capsule, sachet, or other container. When the excipient serves as a diluent, it is a solid, semi-solid, or liquid material that acts as a vehicle, carrier or medium for the synergistic medicament of the present invention. Examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhyclioxybenzoates; sweetening agents; and flavoring agents. The compositions of the invention can be formulated to provide quick, sustained or delayed release of the active ingredient after administration to the host by procedures known in the art. Infusions may be oil based, e.g. a vegetable oil such as peanut oil or a non-vegetable oil such as mineral oil and may further include a thickening agent and optionally also a surfactant. Compositions of the present invention can be in the form of tablets, enteric coated pills, pills, powders, lozenges, sachets, elixirs, suspensions, emulsions, solutions, infusions, syrups, aerosols, ointments, soft and hard gelatin capsules, suppositories, sterile injectable solutions and sterile packages powders. Regardless of the route of administration selected, these compositions are formulated into pharmaceutically acceptable dosage forms by conventional methods known to those skilled in the art.

Actual method of preparing such dosage forms are known, or will be apparent, to those skilled in this art; for example, see Remington's Pharmaceutical Sciences, 18^{th} Ed., (Mack Publishing Company, Easton, Pa., 1990. The composition to be administered will, in any event, contain a therapeutically effective amount of a compound of the invention, or pharmaceutically compositions thereof, for treatment of a disease-state associated with the cancer proliferation activity, in accordance with the teachings of this invention.

The pharmaceutical composition of the present invention in solid or liquid form may include or not include an agent that binds to the compound of the invention and thereby assists in the delivery of the compound. Suitable agents that may act in this capacity include a monoclonal or polyclonal antibody, a protein or a liposome.

The pharmaceutical composition of the present invention may consist of dosage units that may be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredient. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions of the present invention may be prepared by methodology well known in the pharmaceutical art. For example, a pharmaceutical composition intended to be administered by injection IS prepared by combining a compound of the invention with sterile, distilled water so as to form a solution. A surfactant is added to facilitate the formation of a homogeneous solution or suspension. Surfactants are compounds that non-covalently interact with the compound of the invention so as to facilitate dissolution or homogeneous suspension of the compound in the aqueous delivery system.

The compounds of the present invention, or their pharmaceutical compositions, are administered in a therapeutically effective amount, which vary depending upon a variety of factors including the activity of the specific compound employed; the metabolic stability and the length of action of the compound; the age, body weight, general health, sex and diet of the patient; the mode and time of administration; the rate of excretion; the drag combination; the severity of the particular disorder or condition of the subject undergoing therapy. Generally, a therapeutically effective daily dose of Frondoside A or another anti-cancer sea cucumber glycoside consists of daily treatment of 10 μg/kg to as much as 1000 μg/kg. A preferred dose consists of 10-100 μg/kg, 100-200 mg/kg, 200 to 400 μg/kg 400-750 μg/kg or 750 to 1000 μg/kg/day. Generally, a therapeutically effective dose of Andrographolide compound consists of daily dosing of up to 10 mg/kg. Preferred dosing of Andrographolide consists of 0.1 to 1 mg/kg/day, 1-2 mg/kg/day, 2-5 mg/kg/day or 5-10 mg/kg/day.

Preferred dosing of the expected synergistic combination of Frondoside A and/or another sea cucumber anti-cancer glycoside with Andrographolide may consist of 10 μg/kg/day Frondoside, or other sea cucumber glycoside with 0.1 μg/kg/day Andrographolide, 100 μg/kg/day Frondoside A together with 1 mg/kg/day Andrographolide, 500 μg/kg/day Frondoside A, or other sea cucumber glycoside, together with 2-5 mg/kg Andrographolide or 2mg/kg Frondoside A, or other sea cucumber glycoside, together with up to 10 mg/kg Andrographolide. Those skilled in the art will recognize that an ideal dosing regimen for the proposed two drug combination must be derived from preclinical and clinical test results.

Administration of the compounds of the present invention, in pure form or in an appropriate pharmaceutical composition, can be carried out via any of the accepted modes of administration of agents for serving similar utilities. The pharmaceutical compositions of the invention can be prepared by combining a salt of the invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, rectal, vaginal, and intranasal. The term “parenteral” as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage unit, where for example, a tablet may be a single dosage unit, and a container of a salt of the invention in aerosol form may hold a plurality of dosage units.

A pharmaceutical composition of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, e.g., inhalatory administration. When intended for oral administration, the pharmaceutical composition is preferably in either solid or liquid form, where semi-solid, semi-liquid, enteric coated capsule, suspension and gel forms are included within the forms considered herein as either solid or liquid.

As a solid composition of the present invention for oral administration, the pharmaceutical composition may be formulated into a powder, granule, enteric coated compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, e.g., a gelatin capsule, it may contain, in addition to materials of the above type, a liquid carrier such as polyethylene glycol or oil.

The pharmaceutical composition may be in the form of a liquid, e.g., an elixir, syrup, solution, emulsion or suspension. The liquid may be for oral administration or for delivery by injection, as two examples. When intended for oral administration, preferred composition contain, in addition to the present salts, one or more of a sweetening agent, preservatives, dye/colorant and flavor enhancer. In a composition intended to be administered by injection, one or more of a surfactant, preservative, wetting agent, dispersing agent, suspending agent buffer, stabilizer and isotonic agent may be included.

The liquid pharmaceutical compositions of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the invention intended for either parenteral or oral administration should contain an amount of a salt of the invention such that a suitable dosage will be obtained. Typically, this amount is at least 0.01% of a compound of the invention in the composition. When intended for oral administration, this amount may be varied to be between 0.1 and about 70% of the weight of the composition. Preferred oral pharmaceutical compositions contain between about 4% and about 50% of the compound of the invention. Preferred pharmaceutical compositions and preparations according to the present invention are prepared so that a parenteral dosage unit contains between 0.01 to 1% by weight of the compound of the invention.

The pharmaceutical composition of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of the invention from about 0.1 to about 90% w/v (weight per unit volume), preferably between 0.1 and 10%.

The pharmaceutical composition of the invention may be intended for rectal administration, in the form, e.g., of a suppository, which will melt in the rectum and release the drug. The composition for rectal administration may contain an oleaginous base as a suitable nonirritating excipient. Such bases include, without limitation, lanolin, cocoa butter and polyethylene glycol. The pharmaceutical composition of the invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the invention in solid or liquid form may include an agent that binds to the compound of the invention and thereby assists in the delivery of the compound. Suitable agents that may act in this capacity include a monoclonal or polyclonal antibody, a protein or a liposome.

The pharmaceutical composition of the invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredient. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions of the invention may be prepared by methodology well known in the pharmaceutical art. For example, a pharmaceutical composition intended to be administered by injection can be prepared by combining a compound of the invention with sterile, distilled water so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

The compounds of the invention, or their pharmaceutical compositions, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drag combination; the severity of the particular disorder or condition; and the subject undergoing therapy. Generally, a therapeutically effective daily dose is from about 0.1 mg to about 20 mg/kg of body weight per day of a compound of the invention or a pharmaceutically compositions thereof; preferably, from about 0.1 mg to about 10 mg/kg of body weight per day; and most preferably, from about 0.1 mg to about 7.5 mg/kg of body weight per day.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof The pharmaceutically therapeutically active compounds and derivatives thereof are typically formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

The composition can contain along with the active ingredient: a diluent such as lactose, sucrose, dicalcium phosphate, carboxymethylcellulose; a lubricant, such as magnesium stearate, calcium stearate and talc; and a binder such as starch, natural gums, such as gum acacia gelatin, glucose, molasses, polyvinylpyrrolidone, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, for example, acetate, sodium citrate, cyclodextrin derivatives, sorbitan monolaurate, triethanolamine sodium acetate, triethanolamine oleate, and other such agents. Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active compound in an amount sufficient to alleviate the symptoms of the treated subject.

Dosage forms or compositions containing the active ingredient of the present invention in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium croscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polylactic acid and others. Methods for preparation of these compositions are known to those skilled in the art.

The active compounds or pharmaceutically acceptable derivatives may be prepared with carriers that protect the compound against rapid elimination from the body, such as time release formulations or coatings. The compositions may include other active compounds to obtain desired combinations of properties. The compounds provided herein, or pharmaceutically acceptable derivatives thereof as described herein, may also be advantageously administered for therapeutic or prophylactic purposes together with another pharmacological agent known in the general art to be of value in treating one or more of the diseases or medical conditions referred to hereinabove, such as diseases or disorders associated with hypoglycemic activity is implicated. It is to be understood that such combination therapy constitutes a further aspect of the compositions and methods of treatment provided herein.

Oral pharmaceutical dosage forms of the present invention are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, preferably capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, manitol and dicalcium phosphate. Glidants include, but are not limited to, colloidal silicon dioxide. Disintegrating agents include cross carmellose sodium, sodium starch glycolate, alginic acid, corn starch, potato starch, bentonite, methylcellulose, agar and carboxymethylcellulose. Coloring agent include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric-coated tablets, because of enteric-coating, resist the action of stomach acid and dissolve or disintegrate in the neutral or alkaline intestines. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar-coated, multiple compressed and chewable tablets. Flavoring and sweetening agents are especially useful in the formation of chewable tablets and lozenges.

Liquid oral dosage forms include aqueous solutions, emulsions, suspensions, solutions amid/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene laurel ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is preferably encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In all embodiments, tablets and capsules formulations may be coated as known by those with skill in the arts in order to modify or sustain dissolution of the active ingredient. Thus, for example, they may be coated with a conventional enterically digestible coating, such as phenylsalicylate, waxes and cellulose acetate phthalate.

Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleafie and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN.®. 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art. The unit-dose parenteral preparations are packaged in an ampoule, a vial or a syringe with a needle. All preparations for parenteral administration must be sterile, as is known and practiced in the art. Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically, a therapeutically effective dosage is formulated to contain a concentration of at least about 0.1% w/w up to about 90% w/w or more, preferably more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

Lyophilized Powders

Of interest herein is also lyophilized powders, which can be reconstituted for administration as solutions, emulsions and other mixtures. They may also be reconstituted and formulated as solids or gels. The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbitol, fructose, corn syrup, xylitol, glycerin, glucose, sucrose or another suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials or lyophilization. Each vial will contain a single dosage (10-1000 mg, preferably 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, preferably 5-35 mg, more preferably about 9-30 mg of lyophilized powder, is added per ml of sterile water or another suitable carrier. The precise amount depends upon the selected compound. Such amount can be empirically determined.

### DESCRIPTION

The present invention is concerned with therapeutic anticancer combinations having a synergistic effect in the treatment of cancers. The combinations generally comprise a dosage form of a drug of the present invention consisting of a combination of a sea cucumber glycoside, preferably Frondoside A or Frondoside B and Andrographolide or an Andrographolide natural or semi-synthetic analog. The therapeutic effectiveness of the combination is greater than that of the dosage of the single components.

### Details

Andrographolide (50 μM) and Frondoside A (between 1 and 3 mM) was assayed for anticancer effects through determining cell viability of the two human, acute leukemia cell lines CCRF-CEM and THP-1. Results (FIG. 3) show that Andrographolide enhances the effects of Frondoside A in human acute leukemia blast cells and in the THP-1 cells, and show inhibitor effects on existing cancer stem cells present. Andrographolide (50 μM) alone reduced cell viability to 54.3±2.8% in CCRF-CEM cells and to 68.5±1.4%. (FIG. 3)

One skilled in the art recognizes that an amount may be considered therapeutically “effective” even if the condition is not totally eradicated or prevented, but it or its symptoms and/or effects are improved or alleviated partially in the subject. The Physician's Desk Reference (PDR) discloses dosages of various known chemotherapeutic agents. The respective dosing regimens and dosages which are therapeutically effective will depend on the particular cancer being treated, the extent of the disease, and other factors related to the patient as determined by those of ordinary skill in the art.

Andrographolide at 1 mM and Frondoside A (between 1 mM and 3 mM) were assayed for anticancer effects on the viability of the two human pancreatic cancer cell lines, AsPC-1 and S2013. Results indicate that Andrographolide also enhances the effects of Frondoside A in human pancreatic cancer cells, and in this AsPC-1 assay, kills remaining cancer stem cells. (FIG. 4) This suggests a general phenomenon applicable to all or many cancers wherein there is a synergy in the combination of Frondoside A and Andrographolide.

Andrographolide at 1 mM and Stickoposide C at 2 mM is assayed for anticancer effects on the viability of AsPC-1 cells and are determined to be synergistic and inhibitory with almost total inhibition compared to the effects of each compound alone at the same mM treatment doses.

The terms “therapeutic” or “treat,” as used herein, refer to products or processes that are intended to produce a beneficial change in an existing condition (e.g., cancerous tissue, tumor size, metastases, etc.) of a subject, such as by reducing the severity of the clinical symptoms and/or effects of the cancer, and/or reducing the duration of the symptoms/effects of a subject.

In use, a therapeutically-effective amount of the therapeutic combination is administered to a subject in need thereof. In some embodiments, a composition comprising a therapeutically-effective amount of the therapeutic combination (i.e., as a single unit dosage) is administered to a subject. In some embodiments, a therapeutically effective amount of the fortified decoction dosage form and a therapeutically effective amount of the chemotherapeutic agent(s) are co-administered to the subject in need thereof The phrase “co-administer” is intended to embrace administration of each agent in a sequential manner as well as co-administration of these agents in a substantially simultaneous manner in doses given separately. Although described herein with respect to therapeutic treatments, the methods can be also applied for clinical research and/or study.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood, that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

The present disclosure concerns pharmaceutical compositions, methods for preparing such compositions, and methods for their use as are known to those skilled in the arts, particularly orally administered dosage forms having active agents with site specific absorption and enteric coats that release at least a portion of the active agents in acidic gastric fluids.

Enteric Coating

Enteric coating of dosage forms that contain drugs is well known in the pharmaceutical sciences literature. Enteric coatings are coatings designed to prevent release of the enteric-coated drug in gastric fluid of the stomach and prevent exposure of the drug to the acidity of the gastric contents while the enteric coated drug composition is in the stomach. After passing from the stomach into the intestine, the enteric coating dissolves and releases the drug into intestinal fluids.

The Food and Drug Administration (FDA) defines drug dosage forms that are enteric coated as “delayed-release” dosage forms. Delayed-release (enteric coated) dosage forms are differentiated from controlled-release or sustained-release dosage forms, which are intended to provide drug input over an extended period of time, thereby reducing administration frequency. FDA guidelines for enteric-coated dosage forms state: “in-vitro dissolution tests for these products should document that they are stable under acidic conditions and that they release the drug only in a neutral medium (e.g., pH 6.8).” For oral administration, the sea cucumber glycoside and andrographolide compound is delivered in an enteric coated tablet or enteric coated capsules or powders and the combination of the agents is found to be greater than the additive effects of the single agents alone.

In a pharmacokinetics and pharmacodynamics study of Frondoside A by oral route in humans, as are known to those skilled in the arts, dosage of 10 milligrams of Frondoside A is delivered in an enteric coated gelatin capsule, as are known to those skilled in the arts and is detected by LC/MS/MS in the blood up to 20 hours later. In a pharmacokinetics and pharmacodynamics study in a huma of Frondoside A by oral route in humans, a dosage of 10 milligrams of Frondoside A in a capsule without the benefit of enteric coated capsule delivery, is not detected by LC/MS/MS in the blood up to 20 hours later.

Types of Cancer Treatable with the Present Invention

The invention is considered applicable in the treatment of virtually all cancers, such as the following: Acute Lymphoblastic Leukemia, Actinic Keratosis, Adult; Acute Lymphoblastic Leukemia, Childhood; Acute Myeloid Leukemia, Adult; Acute Myeloid Leukemia, Childhood; Adrenocortical Carcinoma; Adrenocortical Carcinoma, Childhood; Adolescents, Cancer in; AIDS-Related Cancers; AIDS-Related Lymphoma; Anal Cancer; Appendix Cancer; Astrocytomas, Childhood; Atypical Teratoid/Rhabdoid Tumor, Childhood, Central Nervous System; Basal Cell Carcinoma; Bile Duct Cancer, Extrahepatic; Bladder Cancer; Bladder Cancer, Childhood; Bone Cancer, Osteosarcoma and Malignant Fibrous Histiocytoma; Brain Stem Glioma, Childhood; Brain Tumor, Adult; Brain Tumor, Brain Stem Glioma, Childhood; Brain Tumor, Central Nervous System Atypical Teratoid/Rhabdoid Tumor, Childhood; Brain Tumor, Central Nervous System Embryonal Tumors, Childhood; Brain Tumor, Astrocytomas, Childhood; Brain Tumor, Craniopharyngioma, Childhood; Brain Tumor, Ependymoblastoma, Childhood; Brain Tumor, Ependymoma, Childhood; Brain Tumor, Medulloblastoma, Childhood; Brain Tumor, Medulloepithelioma, Childhood; Brain Tumor, Pineal Parenchymal Tumors of Intermediate Differentiation, Childhood; Brain Tumor, Supratentorial Primitive Neuroectodermal Tumors and Pineoblastoma, Childhood; Brain and Spinal Cord Tumors, Childhood (Other); Breast Cancer ; Breast Cancer and Pregnancy; Breast Cancer, Childhood; Breast Cancer, Male; Bronchial Tumors, Childhood; Burkitt Lymphoma; Carcinoid Tumor, Childhood; Carcinoid Tumor, Gastrointestinal; Carcinoma of Unknown Primary; Central Nervous System Atypical Teratoid/Rhabdoid Tumor, Childhood; Central Nervous System Embryonal Tumors, Childhood; Central Nervous System (CNS) Lymphoma, Primary; Cervical Cancer; Cervical Cancer, Childhood; Childhood Cancers; Chordoma, Childhood; Chronic Lymphocytic Leukemia; Chronic Myelogenous Leukemia; Chronic Myeloproliferative Disorders; Colon Cancer; Colorectal Cancer, Childhood; Craniopharyngioma, Childhood; Cutaneous T-Cell Lymphoma; Embryonal Tumors, Central Nervous System, Childhood; Endometrial Cancer; Ependymoblastoma, Childhood; Ependymoma, Childhood; Esophageal Cancer; Esophageal Cancer, Childhood; Esthesioneuroblastoma, Childhood; Ewing Sarcoma Family of Tumors; Extracranial Germ Cell Tumor, Childhood; Extragonadal Gem). Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Gastrointestinal Stromal Tumor (GIST); Gastrointestinal Stromal Cell Tumor, Childhood; Germ Cell Tumor, Extracranial, Childhood; Germ Cell Tumor, Extragonadal; Germ Cell Tumor, Ovarian; Gestational Trophoblastic Tumor; Glioma, Adult; Glioma, Childhood Brain Stem; Hairy Cell Leukemia; Head and Neck Cancer; Heart Cancer, Childhood; Hepatocellular (Liver) Cancer, Adult (Primary); Hepatocellular (Liver) Cancer, Childhood (Primary); Histiocytosis, Langerhans Cell; Hodgkin Lymphoma, Adult; Hodgkin Lymphoma, Childhood; Hypopharyngeal Cancer; Intraocular Melanoma; Islet Cell Tumors (Endocrine Pancreas); Kaposi Sarcoma; Kidney (Renal Cell) Cancer; Kidney Cancer, Childhood; Langerhans Cell Histiocytosis; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoma, AIDS-Related; Lymphoma, Burkitt; Lymphoma, Cutaneous T-Cell; Lymphoma, Hodgkin, Adult; Lymphoma, Hodgkin, Childhood; Lymphoma, Non-Hodgkin, Adult; Lymphoma, Non-Hodgkin, Childhood; Lymphoma, Primary Central Nervous System (CNS); Macroglobulinemia, Waldenstrom; Malignant Fibrous Histiocytoma of Bone and Osteosarcoma; Medulloblastoma, Childhood; Medulloepithelioma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndromes, Childhood; Multiple Myeloma/Plasma Cell Neoplasm; Mycosis Fungoides; Myelodysplastic Syndromes; Myelodysplastic/Myeloproliferative Neoplasms; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin Lymphoma, Adult; Non-Hodgkin Lymphoma, Childhood; Non-Small Cell Lung Cancer; Oral Cancer, Childhood; Oral Cavity Cancer, Lip and; Oropharyngeal Cancer; Osteosarcoma and Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell Tumors; Papillomatosis, Childhood; Paranasal Sinus and Nasal Cavity Cancer; Parathyroid Cancer; Penile Cancer; Pharyngeal Cancer; Pineal Parenchymal Tumors of Intermediate Differentiation, Childhood; Pineoblastoma and Supratentorial Primitive Neuroectodermal Tumors, Childhood; Pituitary Tumor; Plasma Cell Neoplasm/Multiple Myeloma; Pleuropulmonary Blastoma, Childhood; Pregnancy and Breast Cancer; Primary Central Nervous System (CNS) Lymphoma; Prostate Cancer; Rectal Cancer; Renal Cell (Kidney) Cancer; Renal Pelvis and Ureter, Transitional Cell Cancer; Respiratory Tract Cancer with Chromosome 15 Changes; Retinoblastoma; Rhabdomyosarcoma, Childhood; Salivary Gland Cancer; Salivary Gland Cancer, Childhood; Sarcoma, Ewing Sarcoma Family of Tumors; Sarcoma, Kaposi; Sarcoma, Soft Tissue, Adult; Sarcoma, Soft Tissue, Childhood; Sarcoma, Uterine; Sezary Syndrome; Skin Cancer (Nonmelanoma); Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Cell Carcinoma; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Testicular Cancer, Childhood; Throat Cancer; Thymoma and Thymic Carcinoma; Thymoma and Thymic Carcinoma, Childhood; Thyroid Cancer; Thyroid Cancer, Childhood; Transitional Cell Cancer of the Renal Pelvis and Ureter; Trophoblastic Tumor, Gestational; Unknown Primary Site, Carcinoma of, Adult; Unknown Primary Site, Cancer of, Childhood; Unusual Cancers of Childhood; Ureter and Renal Pelvis, Transitional Cell Cancer; Urethral Cancer; Uterine Cancer, Endometrial; Uterine Sarcoma; Vaginal Cancer; Vaginal Cancer, Childhood; Vulvar Cancer; Waldenstrom Macroglobulinemia; Wilms Tumor; Women's Cancers.

### REFERENCES

1. Li C, Heidt D G, Dalerba P. Identification of pancreatic cancer stem cells. Cancer Res. 67: 1030-1037, 2007.

2. Hermann P C, Huber S L, Herrler T, et al. Distinct populations of cancer stem cells determine tumor growth and metastatic activity in human pancreatic cancer. Cell Stem Cell. 1: 313-323, 2007.

3. Junfang Ji, Wang X W. Clinical Implications of Cancer Stem Cell Biology in Hepatocellular Carcinoma. Seminars in Oncology 39: 461-472, 2012.

4. Bonnet D, Dick JE. Human acute myeloid leukemia is organized as a hierarchy that originates from a primitive hematopoietic cell. Nat Med 3: 730-737, 1997.

5. Valent P, Bonnet D, De Maria R, et al. Cancer stem cell definitions and terminology: the devil is in the details. Nat Rev Cancer 12: 767-75, 2012.

6. Zhang G N, Liang Y, Zhou L J et al. Combination of salinomycin and gemcitabine eliminates pancreatic cancer cells. Canc Lett. 313: 137-144, 2011.

7. Nusse R. Wnt signaling and stem cell control. Cell Res. 18: 523-527, 2008.

8. Sachloss E, Risueno R M, Laronde S, et al. Idenitification of drugs including a dopamine receptor antagonist that selectively target cancer stem cells. Cell. 149: 1284-1297, 2012.

9. Naujokata C, Lauferc S. Targeting cancer stem cells with defined compounds and drugs. J Canc Res Update 2: 36-67, 2013.

10. Rattan R, Fehmi R A, Munkarah A. Metformin: an emerging new therapeutic option for targeting cancer stem cells and metastasis. J Oncol, 2012; 928217: 1-12

11. Rickenbaugh AL, Baldwin AS. The NF-κB pathway in cancer stem cells. Cells. 2016; 5. 16.

12. Vazquez-Santillan K, Melendez-Zajgla J, Jimenez-Hernandez L, Martinez-Ruiz G, Maldenado V. NF-κB signaling in cancer stem cells: a promising therapeutic target. Cell Oncol. 2015; 38:327-339.

13. Radtke F, Raj K. The role of notch in tumorigenesis: oncogene or tumor suppressor gene? Nat Rev Canc. 3: 756-767, 2003.

14. Li X, Roginsky A B, Ding X Z, Woodward C, Collin P, Newman R A, Bell R H Jr, Adrian T E. Review of the apoptosis pathways in pancreatic cancer and the anti-apoptotic effects of the novel sea cucumber compound, Frondoside A. Aim N Y Acad Sci. 2008; 1138: 181-198.

15. Al Marzouqi N, Iratni R, Nemmar A, Arafat K, Ahmed Al Sultan M, Yasin J, Collin P, Mester J, Adrian T E, Attoub S. Frondoside A inhibits human breast cancer cell survival, migration, invasion and the growth of breast tumor xenografts. Eur J Pharmacol. 2011; 668: 25-34.

16. Attoub S, Arafat K, Gelaude A, Al Sultan M A, Bracke M, Collin P, Takahashi T, Adrian T E, De Wever O. Frondoside a suppressive effects on lung cancer survival, tumor growth, angiogenesis, invasion, and metastasis. PLoS One. 2013; 8: e53087.

17. Park S Y, Kim Y H, Kim Y, Lee S J. Frondoside A has an anti-invasive effect by inhibiting TPA-induced MMP-9 activation via NF-κB and AP-1 signaling in human breast cancer cells. Int J Oncol. 2012; 41: 933-940.

18. Ma X, Kundu N, Collin P D, Goloubeva O, Fulton A M. Frondoside A inhibits breast cancer metastasis and antagonizes prostaglandin E receptors EP4 and EP2. Breast Cancer Res Treat. 2012; 132: 1001-1008.

19. Jin J O, Shastina V V, Shin S W, Xu Q, Park J I, Rasskazov V A, Avilov S A, Fedorov S N, Stonik V A, Kwak J Y. Differential effects of triterpene glycosides, Frondoside A and cucumarioside A2-2 isolated from sea cucumbers on caspase activation and apoptosis of human leukemia cells. FEBS Lett. 2009; 583: 697-702.

20. Al Shemaili J, Mensah-Brown E, Parekh K, Thomas S A, Attoub S, Hellman B, Nyberg F, Adem A, Collin P, Adrian T E. Frondoside A enhances the antiproliferative effects of gemcitabine in pancreatic cancer. Eur J Cancer. 2014; 50:1391-1398.

21. Menchinskaya E S, Aminin D L, Avilov S A, Silchenko A S, Andryjashchenito P V, Kalinin V I, Stonik V A. Inhibition of tumor cells multidrug resistance by cucumarioside A2-2, Frondoside A and their complexes with cholesterol. Nat Prod Commun. 2013; 8:1377-1380.

22. Aminin D L, Agafonova I G, Kalinin V I, Silchenko A S, Avilov S A, Stonik V A, Collin P D, Woodward C. Immunomodulatory properties of Frondoside A, a major triterpene glycoside from the North Atlantic commercially harvested sea cucumber Cucumaria frondosa. J Med Food. 2008; 11: 443-453.

23. Nguyen B C Q, Yoshimura K, Kumazawa S, Tawata S, Maruta H. Frondoside A from sea cucumber and nymphaeols from Okinawa propolis: Natural anti-cancer agents that selectively inhibit PAK1 in vitro. Drug Disc Therapeut (DOI: 10.5582/ddt.2017.01011

24. Huang D B, Vu D, Ghosh G. NF-kappaB RelB forms an intertwined homodimer. Structure. 2005; 13: 1365-1373.

25. Vu D, Huang D B, Vemu A, Ghosh G. A structural basis for selective dimerization by NF-kappaB RelB. J Mol Biol 2013; 425: 1934-1945.

26. Derudder E, Dejardin E, Pritchard L L, Green D R, Korner M, Baud V. Re1B/p50 dimers are differentially regulated by tumor necrosis factor-alpha and lymphotoxin-beta receptor activation: critical roles for p100. J Biol Chem. 2003; 278: 23278-23284.

27. Vallabhapurapu S, Matsuzawa A, Zhang W, Tseng P H, Keats J J, Wang H, Vignali D A, Bergsagel P L, Karin M. Non-redundant and complementary functions of TRAF2 and TRAF3 in a ubiquitination cascade that activates NIK-dependent alternative NF-kappaB signaling. Nat Immunol. 2008; 9: 1364-1370.

28. Jayakumar T, Hsieh C-Y, Lee J-J, Sheu J-R. Experimental and Clinical Pharmacology of Andrographis paniculata and Its Major Bioactive Phytoconstituent Andrographolide. Ev-Based Complem Alt Med. 2013; ID 846740.

29. Burke J R, Pattoli M A, Gregor K R, Brassil P J, MacMaster J F, McIntyre K W, Clarke W, Stranad J, Qiu Y, Zusi FC. BMS-345541 is a highly selective inhibitor of I kappa B kinase that binds at an allosteric site of the enzyme and blocks NF-kappa B-dependent transcription in mice. J Biol Chem. 2003; 278:1450-1456.

We claim:

1. A method of treating cancer, comprising: providing a subject with cancer or a pre-cancer, Frondoside A and an andrographolide compound in an effective therapeutic amount.

2. An anti-cancer synergistic composition of matter comprised of one or more sea cucumber glycosides and an andrographolide compound.

3. An anti-cancer synergistic composition of matter of claim 2, wherein Frondoside A is the sea cucumber glycoside and Andrographolide is the andrographolide compound.

4. A method of treating cancer of claim 1 wherein the andrographolide compound is chosen from the list containing:
a. Neoandrographolide
b. 14-Deoxy-11, 12-didehydroandrographolide
c. deoxyandrographolide
d. Andrographiside
e. Andrograpanin
f. Dehydroandrographolide
g. Andrographolide

5. A method of treating cancer of claim 1 wherein delivery method of said composition of matter is oral and is protected from loss of effectiveness throughout the gastro-intestinal tract in a mammal by an enteric capsule or coating.

6. A composition of matter of claim 1 wherein the precancer is actinic keratosis

7. The composition of matter of claim 2, wherein said sea cucumber glycoside compound is chosen from the list comprising:
1. Cucumarioside A2-2 , A1, A3, A4, A5, A6, A12, and A15
2. Cucumarioside H3, H4
3. Stichoposide C
4. Okhotoside B3
5. Okhotoside B2, B1
6. Intercedenside A
7. Colochiroside A
8. 24-dehydro echinoside
9. Echinoside A
10. Ds-echinoside A
11. Holothurin A
12. Patagonicoside A
13. Philinopside E
14. Frondoside A, B or C
15. Pilinopside A and B
16. Bivittoside A
17. Hillaside B
18. Bivittoside A,
19. Scabraside D,
20. fuscocineroside C
21. 24-dehydroechinoside A
22. Cucumarioside H3,
23. 3-O-methyl-D-xylose, Hillaside A,
24. 17-dehydroxyholothurinoside A,
25. Griseaside A
26. Griseaside D1, Dx and E
27. Philinopgenin A
28. Philinopgenin B
29. Philinopgenin C
30. Synallactosides A1, A2, B1, B2 and C
31. Pentactasides I, II, III
32. Violaceusides A and B
33. Stichorrenoside C and B
